(19) United States
(12) Patent Application Publication
(10) Pub. No.: US 2022/0288132 A1
(43) Pub. Date: Sep. 15, 2022

(54) METHOD FOR PREPARATION OF BLASTOCYST

(71) Applicant: Shingo Miyamoto, Fukuoka (JP)

(72) Inventors: Shingo MIYAMOTO, Kasuga-shi (JP); Katsuro TACHIBANA, Fukuoka-shi (JP); Toyofumi HIRAKAWA, Fukuoka-shi (JP); Fumitoshi KOGA, Fukuoka-shi (JP)

(73) Assignee: Shingo MIYAMOTO, Fukuoka (JP)

(21) Appl. No.: 17/690,559

(22) Filed: Mar. 9, 2022

(30) Foreign Application Priority Data
Mar. 10, 2021 (JP) 2021-038411

(51) Int. Cl. A61K 35/54; C12N 5/073

(52) U.S. Cl. CPC A61K 35/54; C12N 5/0604; C12N 2502/1382; C12N 2506/04

(57) ABSTRACT

Provided a method for preparing a blastocyst. The method includes culturing a fertilized egg in a culture medium containing bubbles containing a reducing gas. The reducing gas may contain at least one selected from the group consisting of hydrogen, carbon monoxide, nitrogen monoxide, and hydrogen sulfide. The bubbles may have an average particle size of from 10 nm to 1,000 nm.

### CROSS-REFERENCE TO RELATED APPLICATION

This application claims priority to Japanese Patent Application No. 2021-038411, filed on Mar. 10, 2021, the content of which is hereby incorporated by reference in its entirety.

### BACKGROUND

### Technical Field

The present disclosure relates to a method for preparation of blastocyst.

### Description of the Related Art

In Japan, one in five married couples is currently diagnosed with infecundity, and the number continues to increase. One of the causes of infertility is implantation failure, which is difficult to treat with the current reproductive medicine technology. For treating implantation failure, for example, regeneration of uterine endometrium, and improvement of the egg quality of a fertilized egg to be implanted are conceivable. In particular, if it is possible to activate maturation of a fertilized egg, such as differentiation induction from an embryo cell to a blastocyst, it can be expected that the implantation rate and therefore the pregnancy rate will be significantly improved.

For example, Japanese Patent Application Laid-Open No. 2016-63804 describes that a composition containing nanobubbles of hydrogen, oxygen, and nitrogen has a cell growth promoting effect.

### SUMMARY

A first embodiment is a method for preparing a blastocyst, the method including culturing fertilized egg in a culture medium containing bubbles containing a reducing gas. The reducing gas may be at least one selected from the group consisting of hydrogen, carbon monoxide, nitrogen monoxide, and hydrogen sulfide. The bubbles may have an average particle size of from 10 nm to 1,000 nm. The fertilized egg may be a human fertilized egg or a fertilized egg of a non-human mammal.

A second embodiment is a method for inducing a fertilized egg into a blastocyst, the method including culturing the fertilized egg in a culture medium containing bubbles containing a reducing gas. The reducing gas may include at least one selected from the group consisting of hydrogen, carbon monoxide, nitrogen monoxide, and hydrogen sulfide. The bubbles may have an average particle size of from 10 nm and 1,000 nm. The fertilized egg may be a human fertilized egg or a fertilized egg of a non-human mammal.

A third embodiment is a cell culture medium containing bubbles containing a reducing gas. The reducing gas may include at least one selected from the group consisting of hydrogen, carbon monoxide, nitrogen monoxide, and hydrogen sulfide. The bubbles may have an average particle size of from 10 nm and 1,000 nm. The cell culture medium may be used for culturing fertilized egg.

### BRIEF DESCRIPTION OF DRAWINGS

FIG. 1 is a graph showing the bubble diameter distribution of carbon monoxide bubbles of Example 1.

FIG. 2 is a graph showing the bubble diameter distribution of oxygen bubbles of Comparative Example 1.

FIG. 3 is a graph showing the differentiation rate (%) of fertilized eggs using a bubble-containing culture medium.

### DETAILED DESCRIPTION

The term “step” includes herein not only an independent step, but also a step which may not necessarily be clearly separated from another step, insofar as an intended function of the step can be attained. In referring herein to the content of a component in a composition, when plural substances exist corresponding to a component in the composition, the content means, unless otherwise specified, the total amount of the plural substances existing in the composition. The upper or lower limit of a numerical range described herein may be optionally replaced with any of limit values of numerical ranges presented as examples. An embodiment of the present invention will be described in detail below. However, the following embodiment is just to present an example of a method for preparing a blastocyst for the sake of embodying the technical idea of the present invention, and the present invention is not limited to the following preparation method.

Method for Preparing Blastocyst

A method for preparing a blastocyst includes a culture process in which a fertilized egg is cultured in a culture medium containing bubbles containing a reducing gas, hereinafter, also referred to as “bubble-containing culture medium”. In vitro culture of a fertilized egg in a culture medium containing a reducing gas as a bubble can efficiently induce differentiation of the fertilized egg into a blastocyst. The development rate or differentiation rates of a fertilized egg or embryo cell can be increased.

The fertilized egg is the first cell to be formed by the union of male and female gametes of an oogamous species. When a fertilized egg is cultured, it differentiates through 2-cell stage embryo, 4-cell stage embryo, 8-cell stage embryo, and morula, and then to blastocyst. The blastocyst is further classified into early blastocyst, mid-stage blastocyst, and late (expanded) blastocyst. When a fertilized egg is differentiation-induced to a blastocyst and then transplanted into the uterus, the implantation rate can be improved in a fertility treatment.

A fertilized egg may be originated from any mammal. Examples of the mammal include human, monkey, pig, cattle, horse, goat, sheep, dog, cat, mouse, rat, guinea pig, and hamster. A fertilized egg may be originated from a human or a nonhuman mammal.

The fertilized egg is cultured in a culture medium containing bubbles containing a reducing gas. The culture medium to be contained bubbles is not particularly restricted as long as the culture medium is capable of culturing a fertilized egg. Examples of the culture medium for a fertilized egg may include KSOM, PAM-5, ORIGIO Sequential Fert (manufactured by ORIGIO), ORIGIO Sequential Blast (manufactured by ORIGIO), ORIGIO Sequential Cleav (manufactured by ORIGIO), SAGE 1-Step (manufactured by ORIGIO), ARTEC (manufactured by Kojin Bio Co., Ltd.), HIGROW OVIT (manufactured by Fuso Pharmaceutical Industries, Ltd.), QA Cleavage Medium (manufactured by Sage BioPharma, Inc.), and Complete Blastocyst Medium (manufactured by Irvine Scientific Sales Co, Inc.) as a culture medium for a late stage, and a mixed culture medium thereof is also applicable. The culture medium may also contain additional additives such as serum, a serum replacement, plasma, serum albumin, a protein, a growth factor, cytokine, a hormone, an amino acid, a vitamin, an antibiotic, etc.

Examples of a gas constituting a bubble contained by a culture medium may include a reducing gas from the viewpoint of the ability of the bubble to induce differentiation into a blastocyst. Examples of a reducing gas may include hydrogen, carbon monoxide, nitrogen monoxide, hydrogen sulfide, and sulfur dioxide, and it is preferable that such a reducing gas contains at least one selected from the group consisting of these gases, and it is more preferable that such a reducing gas contains at least one selected from the group consisting of hydrogen, carbon monoxide, nitrogen monoxide, and hydrogen sulfide. A gas constituting a bubble may further contain nitrogen, oxygen, carbon dioxide, etc., and may also further contain a low molecular hydrocarbon having 5 or less carbon atoms such as methane, ethane, propane, butane, pentane, cyclopropane, cyclobutane, cyclobutane, ethylene, propylene, propadiene, butene, acetylene, propane, propine, etc. A bubble may be composed of only one kind of gas, or a combination of two or more kinds of gases. The content of a reducing gas in a gas constituting a bubble may be, for example, 60% by volume or more, preferably 80% by volume or more, 90% by volume or more, or 95% by volume or more, or the bubble may be substantially composed only of the reducing gas. Herein, “substantially” means that unavoidably contaminated other gases other than a reducing gas are not excluded, and specifically means that the content of the other gases is less than 5 vol %, less than 1 vol %, or less than 0.1 vol %.

Bubbles contained by a culture medium may be ultrafine bubbles or nanobubbles, and the average particle size may be, for example, from 10 nm to 1,000 nm, preferably from 50 nm to 500 nm, or from 100 nm to 300 nm. The average particle size of the bubbles may be, for example, from 10 nm to 100 nm, and preferably from 10 nm to 50 nm. The content (number) of bubbles contained by a culture medium may be, for example, 1×10⁷ (particles/ml) or more, preferably 1×10⁸ particles/ml or more, 5×10⁸ particles/ml or more, or 1×10⁹ particles/ml or more. The upper limit of the number of bubbles contained by a culture medium may be, for example, 1×10¹¹ bubbles/ml or less, and preferably 1×10¹⁰ bubbles/ml or less.

The average particle size of bubbles can be measured, for example, by laser diffraction and scattering, nanoparticle tracking analysis, electrical resistivity, AFM (Atomic Force Microscope), and laser microscope observations. Examples of measurement devices using the laser diffraction and scattering method include a flow cytometer (product name: CytoFlex) manufactured by Beckman Coulter, Inc. Examples of measurement devices using the nanoparticle tracking analysis method include a nanoparticle analysis system (product name: Nonosight) manufactured by Malvern, Inc. For example, a resonant particle measurement system (trade name: Archimedes) manufactured by Malvern can be used as an AFM measurement system.

Examples of the technique for generating bubbles in a culture medium include a swirling liquid flow type, a static mixer type, a venturi type, a pressure dissolution type, and a pore type. Bubbles may be generated in a culture medium by injecting a culture medium into a production container and vibrating the production container in a state in which a desired gas is enclosed.

Specifically, bubbles may be generated in a culture medium as follows. The method of generating bubbles in a culture medium may include preparing a production container, injecting a culture medium to a predetermined height of the production container, sealing the production container while filling the production container with a desired gas, and vibrating the production container at a predetermined rotational speed.

As a production container, a production container composed of a container body including an opening and accommodating a culture medium and a lid capable of sealing the container body is prepared. The container body may, for example, have a bottomed cylindrical shape. Specifically, for example, a vial bottle with a capacity of from 0.5 ml to 20 ml can be used. The dimensions of a vial bottle may be, for example, a longitudinal length X of approximately from 35 mm to 60 mm and an outer diameter of approximately from 10 mm to 40 mm. The lid may include: a disk-shaped rubber stopper (septum) adhering to the opening of the container body; and a tightening portion fixing the rubber stopper to the container body. The rubber stopper may be, for example, a silicone rubber stopper. The tightening portion may be configured to cover an edge of the rubber stopper, and may include an opening approximately centered in the plan view thereof.

To the container body, a culture medium is injected up to a predetermined height. In a state in which the container body in which the culture medium is injected is left standing still horizontally, when the height (length in the longitudinal direction) of the container body is X [mm] and the height of the liquid surface of the culture medium in the container body is Y [mm], the relationship 0.2≤Y/X≤0.7 may be satisfied. In this state, there will be a void portion of sufficient size above the culture medium accommodated in the container body. By vibrating the production container in this state, the culture medium can impinge more vigorously on the top and bottom surfaces and sides of the production container. This impact creates shock waves in the culture medium, which can easily form bubbles in the culture medium.

The container body is filled with the culture medium and sealed with a desired gas. Specifically, the void portion of the container body in which the culture medium has been injected is purged with a desired gas, and then a lid is tightened on the opening of the container body. This seals the culture medium and the desired gas in the production container. Examples of the method of purging a void portion of a container body with a desired gas include a method in which the container body in which a culture medium is injected is moved into a chamber, air in the chamber is replaced with the desired gas, and then a lid is tightened on the opening of the container body. The sealed production container may be pressurized with the desired gas. The sealed production container may be pressurized by adding a desired gas into the sealed production container using a syringe or the like.

By vibrating a production container in which a culture medium and a desired gas are sealed, bubbles are generated in the culture medium. The vibration of the production container may be, for example, a reciprocating motion in the approximate longitudinal direction of the production container. This causes the culture medium to move up and down within the production container and repeatedly impact the top and bottom surfaces and sides of the production container. When the culture medium impinges on the inner surface of the production container, shock waves are generated in the culture medium, and the pressure causes the gas to be finely dispersed in the culture medium, forming bubbles. The frequency of oscillation may be, for example, 5,000 rpm or higher, preferably from 6,000 rpm to 20,000 rpm, or from 6,000 rpm to 7,000 rpm.

The vibration width of a production container in the longitudinal direction may be, for example, approximately from 0.7X [mm] to 1.5X [mm], and preferably from 0.8X [mm] to 1X [mm]. The time for vibrating the production container may be, for example, approximately from 10 seconds to 120 seconds, and preferably from 30 seconds to 90 seconds. By setting the vibration time within the above-described range, the number of times the culture medium impinges on the production container is sufficiently large, and therefore, a large amount of bubbles may be generated in the culture medium. By setting a longer vibration time within the above-described range, the amount of bubbles generated in the culture medium can be increased. Vibration of the production container may be carried out by dividing the vibration time. For example, vibration of the production container may be carried out by repeating vibration of from 5 to 30 seconds for approximately 3 to 10 times.

For example, a bead-based high-speed cell disruption system (homogenizer) may be used as a device capable of vibrating the production container. Specifically, Precellys (^{R}) manufactured by Bertin Technologies Inc. or the like may be used. Details of the method of generating bubbles in the culture medium can be found, for example, in the description of International Publication No. WO2016/163439, the content of which is hereby incorporated by reference in its entirety.

In the method for preparing a blastocyst, a fertilized egg is cultured in a bubble-containing culture medium. The culture conditions for a fertilized egg may be generally employed conditions. For example, in the case of a human fertilized egg, they may be 37° C., and 5% CO₂. In the case of that of a pig, cattle, horse, or the like, they may be, for example, from 38° C. to 39° C., and 5% CO₂. There is no particular restriction on the incubation time as long as a blastocyst is formed, for example, from 2 days to 10 days. The number of fertilized eggs to be cultured per each vessel may be one, or may be plural.

Another embodiument of the present invention may be a method for inducing a fertilized egg to a blastocyst, or a method for inducing differentiation, which includes culturing a fertilized egg in a culture medium containing bubbles containing a reducing gas. Another embodiment may be a method for producing a blastocyst, including culturing a fertilized egg in a culture medium containing bubbles containing a reducing gas.

Bubble-Containing Cell Culture Medium

The bubble-containing cell culture medium includes a cell culture medium and bubbles containing a reducing gas present in the cell culture medium. The bubbles contained in the bubble-containing cell culture medium may be ultrafine bubbles or nanobubbles. By culturing a cell in a bubble-containing cell culture medium, the development rate of the cultured cell can be enhanced. By culturing a fertilized egg in a bubble-containing cell culture medium, the fertilized egg can be induced to differentiate into a blastocyst with excellent efficiency, and the development rate or the differentiation rate of the fertilized egg or embryo cell may be enhanced.

The bubble-containing cell culture medium may be prepared by generating bubbles containing a reducing gas in a usually used cell culture medium. The details of gases constituting the bubbles and the method for generating the bubbles are as described above.

The cell culture medium may be any usual culture medium for animal cell culture, or may be the above-described culture medium for fertilized egg. Examples of the culture medium for animal cell culture may include Dulbecco's modified Eagle's medium (DMEM) (Nissui Pharmaceutical Co. Ltd. etc.), α-MEM (Dainippon Pharmaceutical Co., Ltd., etc.), DMEM: Ham' F12 mixed medium (1:1) (Dainippon Pharmaceutical Co. Ltd. etc.), Ham's F12 medium (Dainippon Pharmaceutical Co., Ltd., etc.), and MCDB201 medium (Functional Peptide Institute). The cell culture medium may further contain an additive such as a serum, a serum substitute, a plasma, a serum albumin, a protein, a growth factor, a cytokine, a hormone, an amino acid, a vitamin, an antibiotic, etc., as needed.

The average particle size of bubbles contained in the cell culture medium may be, for example, from 10 nm to 1,000 nm, preferably from 50 nm to 500 nm, or from 100 nm to 300 nm. The average particle size of the bubbles may be, for example, from 10 nm to 100 nm, and preferably from 10 nm to 50 nm. The content (number) of bubbles contained in the cell culture medium may be, for example, 1×10⁷ (particles)/ml or more, preferably 1×10⁸/ml or more, 5×10⁸/ml or more, or 1×10⁹/ml or more. The upper limit of the number of bubbles contained in the culture medium may be, for example, 1×10¹¹ bubbles/ml or less, and preferably 1×10¹⁰ bubbles/ml or less.

The cell to which the bubble-containing cell culture medium is applied is not particularly restricted to any species or origin tissue as long as the cell is an animal cell. The cell may be either a somatic cell, a fertilized egg, a stem cell, or the like. The cell may be a cell immediately after harvesting from a living body, a dendritic cell line, or the like. The animal cell may be of any mammalian origin, including a human being. Preferably, the bubble-containing cell culture medium may be used to culture a fertilized egg.

Treatment Method for Infertility

A treatment method for infertility is a method for treating infertility in a subject, the method including: inducing differentiation of a fertilized egg into a blastocyst by culturing the fertilized egg in a culture medium containing bubbles containing a reducing gas; and transplanting the differentiation-induced blastocyst into the uterus of a subject. The implantation rate is improved by transplanting a differentiation-induced blastocyst. Any mammal may be the target animal of the treatment method for infertility, and the mammal includes a human. The target subject may also be a non-human mammal. The method for treating infertility may further include: collecting an unfertilized egg from a subject; and fertilizing the unfertilized egg to obtain a fertilized egg.

Improvement of Implantation Failure

An improvement method for implantation failure is a method for treating implantation failure in a subject, the method including: inducing differentiation of a fertilized egg into a blastocyst by culturing the fertilized egg in a culture medium containing bubbles containing a reducing gas; and transplanting the differentiation-induced blastocyst into the uterus of a subject. The implantation rate is improved by⁻transplanting a differentiation-induced blastocyst. Any mammal may be the target animal of the improvement method for implantation failure, and the mammal includes a human. The target subject may also be a non-human mammal. The improvement method for implantation failure may further include: collecting an unfertilized egg from a subject; and fertilizing the unfertilized egg to obtain a fertilized egg.

Kit for Culturing Fertilized egg

A kit for culturing a fertilized egg may include: the above-described bubble-containing cell culture medium; and instructions for use that describe culturing a fertilized egg in the bubble-containing cell culture medium to induce differentiation of the fertilized egg into a blastocyst.

### EXAMPLES

The present invention will be more specifically described below by way of Examples, provided that the present invention is not limited to such Examples.

### Example 1

KSOM was prepared as a fertilized egg culture medium. One ml of KSOM was placed in a vial bottle with a capacity of 2 ml, purged with carbon monoxide, and sealed by plugging a lid. Two ml of carbon monoxide was added to the vial bottle using a syringe. The sealed vial bottle was vibrated six times at 6,500 rpm for 10 seconds using a Precellys (^{R}) (high speed cell disruption system) manufactured by Bertin Technologies Inc. to obtain a culture medium containing carbon monoxide bubbles.

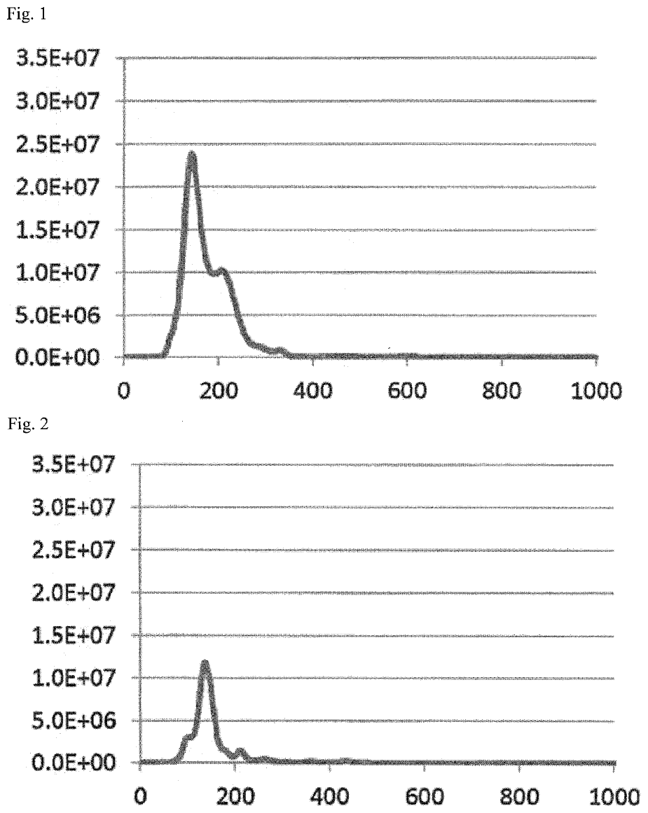

A nanoparticle analysis system (NanoSight; manufactured by Malvern, Inc.) was used to measure the bubble diameter distribution of bubbles in the bubble-containing culture medium. The results are shown in FIG. 1. The average particle size of the bubbles was 175 nm and the mode diameter was 142 nm. The content of the bubbles was 1.9×10⁹ bubbles/ml.

Comparative Example 1

A culture medium containing oxygen bubbles was obtained in the same manner as in Example 1, except that oxygen was used instead of carbon monoxide.

The results of the measurement of bubble diameter distribution are shown in FIG. 2. The average size of the bubbles was 150 nm and the mode diameter was 137 nm. The content of the bubbles was 5.9×10⁸ bubbles/ml.

Evaluation

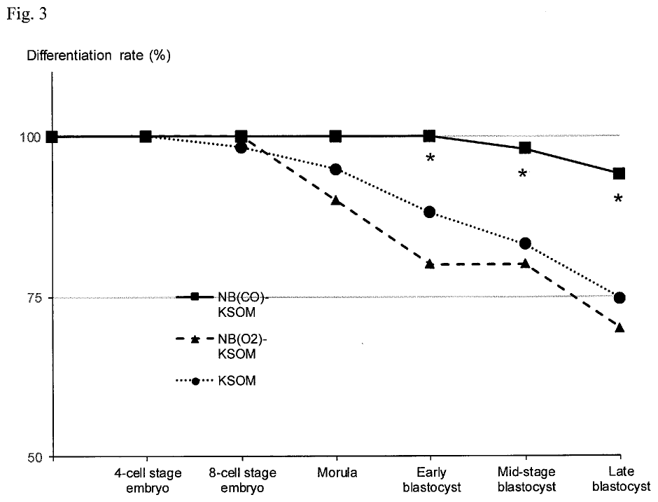

Using the culture medium obtained in Example 1 (NB(CO)-KSOM), the culture medium obtained in Comparative Example 1 (NB(O₂)-KSOM), and the culture medium without bubble formation (KSOM), the differentiation state of the fertilized egg was evaluated as follows. A droplet of a culture medium was formed in a cell culture dish, and a 2-cell stage embryo of an ICR mouse was seeded in the droplet and incubated at 37° C. and 5% CO₂. Observations were made daily until 96 hours after the start of culture, and the differentiation rates (%) of a 4-cell stage embryo, an 8-cell stage embryo, a morula, an early blastocyst, a mid-stage blastocyst, and a late blastocyst were calculated, respectively. The results are shown in FIG. 3 and Table 1. In FIG. 3, * indicates a significant difference (p<0.05) between NB(CO)-KSOM and KSOM. Significant differences were tested with the Mann-Whitney U test.

TABLE 1

| | NB(CO)-KSOM | NB(O₂)-KSOM | KSOM |
|---|---|---|---|
| | (%) | (%) | (%) |
| 4-cell stage embryo | 100.0 | 100.0 | 100.0 |
| 8-cell stage embryo | 100.0 | 100.0 | 98.3 |
| Morula | 100.0 | 90.0 | 94.9 |
| Early blastocyst | 100.0 | 80.0 | 88.1 |
| Mid-stage blastocyst | 98.0 | 80.0 | 83.1 |
| Late blastocyst | 94.0 | 70.0 | 74.6 |

As shown in Table 1 and FIG. 3, by culturing a fertilized egg in a culture medium containing bubbles of a reducing gas, the fertilized egg differentiated efficiently.

It is to be understood that although the present invention has been described with regard to preferred embodiments thereof, various other embodiments and variants may occur to those skilled in the art, which are within the scope and spirit of the invention, and such other embodiments and variants are intended to be covered by the following claims.

Although the present disclosure has been described with reference to several exemplary embodiments, it is to be understood that the words that have been used are words of description and illustration, rather than words of limitation. Changes may be made within the purview of the appended claims, as presently stated and as amended, without departing from the scope and spirit of the disclosure in its aspects. Although the disclosure has been described with reference to particular examples, means, and embodiments, the disclosure may be not intended to be limited to the particulars disclosed; rather the disclosure extends to all functionally equivalent structures, methods, and uses such as are within the scope of the appended claims.

One or more examples or embodiments of the disclosure may be referred to herein, individually and/or collectively, by the term “disclosure” merely for convenience and without intending to voluntarily limit the scope of this application to any particular disclosure or inventive concept. Moreover, although specific examples and embodiments have been illustrated and described herein, it should be appreciated that any subsequent arrangement designed to achieve the same or similar purpose may be substituted for the specific examples or embodiments shown. This disclosure may be intended to cover any and all subsequent adaptations or variations of various examples and embodiments. Combinations of the above examples and embodiments, and other examples and embodiments not specifically described herein, will be apparent to those of skill in the art upon reviewing the description.

In addition, in the foregoing Detailed Description, various features may be grouped together or described in a single embodiment for the purpose of streamlining the disclosure. This disclosure may be not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The above disclosed subject matter shall be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure may be determined by the broadest permissible interpretation of the following claims and their equivalents, and shall not be restricted or limited by the foregoing detailed description.

All publications, patent applications, and technical standards mentioned in this specification are herein incorporated by reference to the same extent as if each individual publication, patent application, or technical standard was specifically and individually indicated to be incorporated by reference.

What is claimed is:

1. A method for preparing a blastocyst, comprising culturing a fertilized egg in a culture medium containing bubbles containing a reducing gas.

2. The preparation method according to claim 1, wherein the reducing gas comprises at least one selected from the group consisting of hydrogen, carbon monoxide, nitrogen monoxide, and hydrogen sulfide.

3. The preparation method according to claim 1, wherein the bubbles have an average particle size of from 10 nm to 1,000 nm.

4. The preparation method according to claim 1, wherein the fertilized egg is a human fertilized egg.

5. The preparation method according to claim 1, wherein the fertilized egg is a fertilized egg of a non-human mammal.

6. A method for inducing a fertilized egg into a blastocyst, the method comprising culturing the fertilized egg in a culture medium containing bubbles containing a reducing gas.

7. A bubble-containing cell culture medium comprising a cell culture medium and a bubble containing a reducing gas present in the cell culture medium.

8. The bubble-containing cell culture medium according to claim 7, wherein the reducing gas comprises at least one selected from the group consisting of hydrogen, carbon monoxide, nitrogen monoxide, and hydrogen sulfide.

9. The bubble-containing cell culture medium according to claim 7, wherein the bubbles have an average particle size of from 10 nm to 1,000 nm.

10. The bubble-containing cell culture medum according to claim 7, which is used for culturing a fertilized egg.

11. A method for treating infertility in a subject, the method comprising: culturing a fertilized egg in a culture medium containing bubbles containing a reducing gas to induce differentiation of the fertilized egg into a blastocyst; and transplanting the differentiation-induced blastocyst into a uterus of a subject.

12. The treatment method according to claim 11, wherein the reducing gas comprises at least one selected from the group consisting of hydrogen, carbon monoxide, nitrogen monoxide, and hydrogen sulfide.

13. The treatment method according to claim 11, wherein the bubbles have an average particle size of from 10 nm to 1,000 nm.

14. The treatment method according to claim 11, wherein the fertilized egg is a human fertilized egg.

15. The treatment method according to claim 11, wherein the fertilized egg is a fertilized egg of a non-human mammal.
